(19) United States
(12) Patent Application Publication
(10) Pub. No.: US 2005/0233015 A1
(43) Pub. Date: Oct. 20, 2005

(54) Lanolin substitute based on shea butter

(76) Inventors: Staffan Norberg, Karlshamn (SE); Jari Alander, Karlshamn (SE); Roger Jacobsson, Karlshamn (SE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/515,188

(22) PCT Filed: Apr. 1, 2003
(86) PCT No.: PCT/SE03/00521

§ 371 Date: Nov. 29, 2004

(30) Foreign Application Priority Data
May 27, 2002 (SE) 0201566-7

(51) Int. Cl. A61K 35/78

(52) U.S. Cl. 424/769

(57) ABSTRACT

The invention refers to a lipid composition with lanolin-like properties, which comprises a combination of non-polar unsaponifiable matter from vegetable oils and fats and a polar mixture of fatty acid esters. The composition can be used as a lanolin substrate in different formulations, especially for cosmetic use.

The present invention refers to a lipid composition, which can be used as a lanolin substitute in formulations of different types, especially for cosmetic use.

### BACKGROUND

Today, lanolin and its derivatives are widely used in many industries, especially in cosmetics and pharmaceuticals. Lanolin has many attractive properties, such as adhesion, hydrating properties, gloss, touch and spreadability. However, the colour, smell and taste are some of the reasons why the cosmetic industry is looking for alternatives.

Lanolin is a natural product with a very complex composition. The main constituents are esters of fatty acids, esters of hydroxy acids, esters of sterols and triterpene alcohols, as well as the free acids, sterols and alcohols. It is the mixture of these compounds that gives lanolin its characteristic properties. Lanolin and derivatives thereof can be fractionated to yield a number of products with different properties with respect to polarity, emulsifying properties, adhesion, melting point and viscosity. Native lanolin can be fractionated into one harder and one fluid fraction, which are referred to as lanolin wax and lanolin oil, respectively.

Vegetable oils such as rapeseed oil, sunflower oil, olive oil, cotton seed oil, safflower oil, soya bean oil, palm oil, palm kernel oil, coconut oil but also more exotic oils such as shea butter, illipe, mango butter and avocado oil are widely used within the food industry as well as in the cosmetic area. The major components in vegetable oils and fats are triglycerides. Vegetable oils and fats are appreciated in the cosmetic industry for their moisturising and re-fattening properties. However, they have no emulsifying properties and have much less adhesion compared to lanolin.

Fats mixed with polyols, for instance glycerol, polyglycerol, sucrose and polyethylene glycol, can undergo alcoholysis when heated. This process is facilitated by catalysts. In the case of fats and glycerol, an intermolecular exchange of fatty acids results in numerous new compounds, such as monoglycerides and diglycerides. When the reaction has reached its equilibrium a mixture of glycerol, triglycerides, diglycerides and monoglycerides is obtained. The mixture obtained has a higher polarity compared to the original fats, but lacks the desired touch and adhesion of lanolin.

### PRIOR ART

Throughout the years, many attempts to imitate lanolin have been made.

U.S. Pat. No. 4,868,220, Henkel Kommanditgesellschaft and Aktien, discloses a substitute for lanolin comprising a mixture of saturated and unsaturated mono- and diglycerides, ethoxylated sterols, and di-fatty acid esters of penta-erythritol. The product obtained is described as having a consistency similar to natural lanolin, and having the advantage of being hypoallergenic and almost odourless. The substitute is used for preparing creams, ointments and lotions.

U.S. Pat. No. 5,436,006, The Nisshin Oil Mills, Ltd, discloses a lanolin-like synthetic oil which is produced by esterification of glycerol with dibasic acids, branched fatty acids and longer fatty acids. Commercial products based on this technology, such as Kahls lanolin substitute (Kahl, Germany), have a poor taste, which is unacceptable in for instance lipstick formulations.

U.S. Pat. No. 5,279,830, Intellectual Property Holding Co., discloses a cosmetic composition free of lanolin, but including the cosmetically acceptable ingredients: a wax, a triglyceride, an ester mixture, distarch phosphate and conventional additives. Said composition is preferably formed into a cosmetic stick.

However, the synthetic substitutes described so far are in addition to being expensive to make also associated with poor taste and dry touch.

### DESCRIPTION OF THE INVENTION

It has now surprisingly been found that a composition based on a combination of natural glycerides of vegetable oils and fats, and unsaponifiable matter can mimic the properties of lanolin without having the inconvenient smell, taste and odour.

Native lanolin is a semi-solid, unctuous mass melting at about 36-42° C. It is insoluble in water and polar solvents but forms readily a w/o emulsion when mixed with water. It can be dissolved in non-polar solvents and is compatible with most types of cosmetic emollients. Lanolin is very adhesive towards skin and hair, has high substantivity and can be used as a thickener and tackifier in different formulations. It is also a good emollient with soothing and lubricating properties.

When comparing the properties of different lanolin substitutes two important properties to evaluate are the consistency and the polarity. The consistency can to some extent be defined by the melting point, the adhesion, and the viscosity of the composition. The polarity is given by the hydroxyl value and also by the water absorbing capacity and the emulsifying capacity.

The present invention refers to a new lipid composition with lanolin-like properties, which is characterised in comprising a combination of (i) non-polar unsaponifiable matter from vegetable oils and fats in an amount not less than 2% by weight and (ii) a polar mixture of fatty acid esters. Said composition should have an adhesiveness and a water absorption of at least 50% by weight.

The water absorption can be determined as the maximum amount of water giving a homogenous emulsion after storage at 30° C. for 72 hours. The emulsion is prepared by heating the lipid component to complete melting for 30 minutes, adding water of 40° C., and shaking at 2000 rpm in a Whirli Vib 1 vibromix for 10 seconds.

The adhesive properties of the lipid composition are obtained from polymers derived from vegetable oils, preferably containing long-chain hydrocarbons, which can be saturated or unsaturated. As examples of natural polymers can be mentioned estolides and isoprenoids. It is likely that also synthetic polymers, such as polyolefins, like polyethylene, polyesters, polyamides, polyacrylates and polypropylene glycols, can be used to provide the adhesive or tackifying properties.

The invention refers to a lipid composition comprising unsaponifiable matter from a vegetable oil. All vegetable oils contain unsaponifiable matter in a greater or less degree. Unsaponifiable matter is defined as the material from a lipid sample, which can be extracted by petroleum ether or diethyl ether after alkaline hydrolysis, as determined according to AOCS Ca-6a40. As examples can be mentioned alcohols, sterols and hydrocarbons. Vegetable oils having a high content of unsaponifiable matter are shea butter, olive oil, and avocado oil, but also other oils such as soya bean oil, rape seed oil are of interest in this connection.

The shea tree (Butyrospermum parkii) grows in the savannah region in West Africa. The crude oil, shea butter, is obtained from the nuts, has an iodine value around 65 and contains triglycerides, mainly of stearic and oleic acids, and a high amount of unsaponifiable matter, up to 13%. The unsaponifiable matter includes a wide range of components, mainly long-chain hydrocarbons, sterols, triterpene alcohols, and esters of sterols and triterpene alcohols. As examples can be mentioned tocopherols, phytosterols, cinnamic acid esters, waxes, and as long-chain hydrocarbons, isoprenes. According to a preferred aspect the invention refers to a lipid composition containing unsaponifiable matter from shea butter.

In olive oil the content of unsaponifiable matter is typically 1-5%. Multiple branched chains in the range of C16-C36, e.g. squalene and isoprenoidal polyolefins are the major constituents of the unsaponifiable fraction, but also some n-alkanes in the range of C13-C30 are present.

To obtain a fraction enriched in unsaponifiable matter, acetone or other suitable solvents is added to the crude or refined vegetable oil, or to some other fraction of the oil. The unsaponifiable fraction is precipitated in the solvent and can be separated from the oil.

The unsaponifiable fraction can then be mixed with a vegetable oil or fat of choice to improve the adhesive properties of the oil.

The fatty acid esters in the polar mixture can be derived from straight or branched, saturated or unsaturated C8 to C24 fatty acids or hydroxy fatty acids. The saturated fatty acids can be naturally saturated acids or hydrogenated unsaturated fatty acids. As examples can be mentioned stearic acid, oleic acid, palmitic acid, caprylic acid, caproic acid, lauric acid, myristic acid, linoleic acid, linolenic acid, arachidic acid, behenic acids, lignoceric acid, arachidonic acids, erucic acids, eicosapentadienoic acid, docosahexadienoic acid, isostearic acid, isomyristic acid, isopalmitic acid, isooleic acids, 12-hydroxy stearic acids, 12-hydroxy ricinoleic acids, 9,10-dihydroxy stearic acids, coriolic acid, allenic hydroxy acids, 2,4-decadienoic acid and monohydroxy tricarboxylic acids.

The invention especially refers to a lipid composition wherein the fatty acid esters are derived from glycerol, polyglycerols or polyethylene glycol. The esters of glycerol can be mono, di or triglycerides or a mixture thereof. Other esters of interest are fatty acid esters of sucrose, mannitol, glucose, pentaerythritol, trimethylolpropane and other polyols.

According to a special aspect of the invention the mixture of fatty acid esters is derived from a vegetable oil by alcoholysis or interesterification. The vegetable oil can be chosen from the group consisting of rapeseed oil, sunflower oil, corn oil, olive oil, cotton seed oil, safflower oil, soya bean oil, palm oil, palm kernel oil, coconut oil, as well as the more exotic oils shea butter, illipe, mango butter, avocado oil, castor oil and stillingia oil.

In order to mimic the properties of lanolin the lipid composition of the invention should preferably have a hydroxyl value of 20-180, a slip melting point of 30-55° C., and a water absorption of 75-200% by weight of the composition.

According to another aspect of the invention the content of unsaponifiable matter in the composition of the invention should be 2-25% by weight of the composition, preferably 5-15%.

The composition of this invention is produced by means of processes used in the oil processing industry, such as alcoholysis, glycerolysis and/or interesterification. The unsaponifiable fraction may either be used in its native form or be part of an alcoholysis, glycerolysis and/or interesterification process. The composition of the invention can be obtained in.several ways. A fraction rich in unsaponifiable matter can be isolated from various sources and be used as such, added to a glycerolised or poly-glycerolised fatty acid ester or added to a triglyceride oil prior to glycerolysis or polyglycerolysis. The unsaponifiable fraction may optionally be hydrogenated to increase its melting point and adhesiveness as well as oxidative stability.

The invention also refers to the use of a lipid composition as described in any formulation as a substitute for lanolin.

Preferably the lipid composition of the invention is used in combination with conventional additives and active substances in a pharmaceutical or cosmetic formulation. As examples of conventional additives can be mentioned emollients, preservatives, colouring agents, and aromas.

The invention also refers to the use of the composition of the invention in colour cosmetics, toiletries, skin care, hair care and bath and shower products.

### EXAMPLES OF PREPARATIONS OF LIPID COMPOSITIONS

In order to evaluate the properties of the different lipid compositions prepared in the following examples the following characteristics were determined.

Hydroxyl value

The hydroxyl value is defined as the number of mg KOH and was determined according to AOCS Cd 13-60.

Saponification value

The saponification value refers to the number of mg of KOH required to saponify one gram of oil or fat, and has been determined by the standard method IUPAC 2,202.

Unsaponifiable matter

The material from a lipid sample which can be extracted by petroleum ether or diethyl ether after alkaline hydrolysis, comprising hydrocarbons, alcohols, and sterols but not water soluble compounds. The content of unsaponifiable matter has been determined in accordance with AOCS Ca-6a40.

Slip melting point

The slip melting point is determined In accordance with AOCS Cc-3-254.

Capillary viscosity

The capillary viscosity is determined in accordance with Astm D445-96 at 50° C.

Viscosity

The Theological properties were measured with Physica UDS 200, 35° C., cone and plate geometry, shear rate 0,1-200 1/s. 1-2 g of each sample was gently transferred to the plate at room temperature. The temperature was rapidly increased to 35° C. The viscosity was evaluated at 35° C. in the shear rate range of 0.1 to 200 s⁻¹.

Hygroscopic capacity

The hygroscopic or water absorbing properties of samples from the examples were compared with lanolin. Three 10 g-samples of the product as well as three 10 g-samples of lanolin and lanolin oil in open cups were kept at 30° C. and 100% RH (relatively humidity) for two weeks. The weights were measured before and after the storage. The water absorbing capacity is expressed as weight increase in % after two weeks at 30° C. and 100% RH.

Iodine value

The iodine value was determined according to IUPAC 2.2054 using a modified Hanus method. This will give the unsaturation of the composition in mg I₂/g fat.

Some of the values obtained were compared to the values of commercial lanolin products. Lanolin refers in this context to Fancor Lanolin, from Fanning Corporation, USA, and Lanolin oil refers to Vigilan, from Fanning Corporation, USA, if nothing else is stated.

In addition tests of the adhesiveness and water absorbing capacity were performed.

### Example 1

### Preparation of Non-polar Unsaponifiable Matter from Shea Butter, Used as Starting Material

(a) A highly concentrated unsaponifiable fraction was separated from crude shea oil by adding acetone to the crude shea oil and precipitating the unsaponifiable matter. The precipitate was separated from the oil and acetone solution by centrifugation. A liquid shea fraction was mixed with the unsaponifiable matter precipitate and the resulting mixture was refined and deodorised according to standard procedures.

The unsaponifiable matter and capillary viscosity at 50° C. were determined to 16.5% and 780 mpas, respectively. The adhesion was extremely high. This product was subsequently used in different compositions and formulations in the following Examples 3b, 4-7.

When this example was repeated with refined shea oil, the precipitate obtained by adding acetone to the refined shea oil was separated from the oil and the acetone solution by centrifugation. A liquid shea fraction was then mixed with the unsaponifiable matter. The product was very adhesive and the content of unsaponifiable matter in the product was 22.4%. The viscosity at 70° C. was 750 mPas. This product was subsequently used in the compositions of the following Examples 8 and 9.

The product obtained in Example 1(a) was hydrogenated lightly at 2,5 bar of hydrogen and 0.1% of nickel catalyst (Nysosel 325, Engelhardt, Bad Wortum, Germany) was added at 150° C. for 2 hours. After removal of the nickel catalyst by filtration, the oil was bleached and deodorised using the procedure as described in Example 2 below. The iodine value was reduced from 77 to 68, and the texture became slightly harder.

The relative oxidation stabilities of the hydrogenated and non-hydrogenated products were determined by differential scanning calorimetry (Mettler TA8000, standard open aluminium crucibles, heating from 20° C. to 250° C. at a rate of 3° C./min, nitrogen purge gas, sample size 5-10 mg). As the temperature reaches a critical value, a thermal breakdown of the product occurs and can be followed by recording the endothermal heat-flow in the sample. The extent of reaction at different temperatures was determined by consecutive partial integrations of the heat-flow curves by the routines available in the calorimeter software and are reported in Table 1. The higher oxidation stability in the hydrogenated product can be seen for example by determining the temperature where 50% of the breakdown reaction has taken place. This temperature is 138° C. for the non-hydrogenated product and approximately 144° C. for the hydrogenated one, which indicates a significantly better oxidative stability for the hydrogenated product.

TABLE 1
Oxidation stability at different temperatures

| | | Hydrogenated product |
|---|---|---|
| | Product of Example 1(a) | of Example 1(a) |
| Temperature | Iodine value 77 | Iodine value 66 |
| 120 | 0 | 0 |
| 125 | 1 | 1 |
| 130 | 17 | 7 |
| 135 | 38 | 19 |
| 137.5 | 50 | 27 |
| 140 | 61 | 37 |
| 142.5 | 71 | 47 |
| 145 | 80 | 57 |
| 150 | 93 | 75 |
| 155 | 100 | 88 |
| 160 | 100 | 97 |
| 165 | 100 | 100 |

### Example 2

### Glycerolysis of Shea Butter

To a 1 litre four-necked flask, 425 g of crude sheabutter (karlshamns AB, Karlshamn, Sweden), characterised by an iodine value of 65 and a content of unsaponifiable matter of 7%, and 75grams of anhydrous glycerol (Tefac AB, Karlshamn, Sweden) were added. The reactants were dried at 100° C. with gentle agitation and a vacuum of 5 mbar for 30 minutes to remove residual water. 1 g of sodium hydroxide (Kebo, Stockholm, Sweden) was added. The temperature was set at 140° C. for two hours to complete the reaction. The temperature was lowered to 90° C. and the reaction was quenched by the addition of 10 ppm of citric acid in ethanol (20% solution). The colour was decreased with a bleaching step where 1% bleaching earth (Tonsil Optimum 215, Sudchemie, Germany) was added to the flask. The bleaching procedure took 30 minutes at 90° C. with agitation and a 5 mbar vacuum. After the completion of the bleaching step, the bleaching earth was filtered away. The product was then heated to 140° C. (3 mbar, 2 h) steam was flushed through the product at a rate of 36 ml/h/kg. After cooling to 90° C., 200 ppm ascorbyl palmitate (Grindox Ascorbyl Palmitate Fine, Danisco, Brabrand, Denmark) was added to the product as an antioxidant. The hydroxyl value, saponification value, unsaponifiable matter, slip melting point and capillary viscosity were determined to 106, 167, 5.7%, 50° C. and 76 mPas, respectively.

### Example 3

### Lipid Composition from Shea

(a) Glycerolysis of a liquid shea butter fraction

A bleached shea butter having a melting point of 34° C. was first fractionated at +4° C. using a temperature gradient of 0.5° C./min and an acetone/oil ratio of 5/1 (v/w) to yield a solid fraction and a filtrate containing a liquid fraction in approximately equal yields. The liquid shea fraction (obtained as described in WO 99/63031), characterised by an iodine value of 75 and an unsaponifiable content of 8%, was glycerolysed by the procedure of Example 2. This time the ratio oil/glycerol was 90:10 (w/w). The hydroxyl value, saponification value, unsaponifiable matter, slip melting point, and capillary viscosity at 50° C. were determined to 109, 167, 7.5%, 40° C. and 49 mpas, respectively.

The Theological properties were measured as described. A sample of the product obtained in this example was compared to lanolin and lanolin oil. At the shear rate 36 1/s, the viscosity of the product of this example was 2.6 Pas, which was lower than the viscosity of lanolin (12.1 Pas) but higher than for lanolin oil (1.1 Pas).

The hygroscopic properties of samples from this example were compared with lanolin. The hygroscopic capacity is expressed as weight increase in % after two weeks at 30° C. and 100% RH. The product of this example increased 5% (±1%) in weight during these two weeks. This was slightly higher than both lanolin (1%) and lanolin oil (2% ±1%).

(b) Mixing of Glycerolised Shea Butter Fraction and Unsaponifiable Fraction from Shea

The product obtained above was mixed with the product of Example 1 and above were mixed (90:10) in order to obtain improved emulsifying properties in combination with high adhesion. The new product was bleached and deodorised by adding 1% bleaching earth (Tonsil Optimum 215 FF, Sudchemie, Germany) mixture, followed by heating to 90° C. with agitation and at a 10 mbar vacuum for 30 minutes. After the completion of the bleaching step, the bleaching earth was filtered away. The product was then heated to 140° C. (3 mbar, 2 hours) and steam was flushed through the product at a rate of 36 ml/h/kg. After cooling to 90° C., 200 ppm ascorbyl palmitate (Grindox Ascorbyl Palmitate Fine, Danisco, Brabrand, Denmark) was added to the product as an antioxidant. The hydroxyl value, saponification value, unsaponifiable matter, slip melting point, and capillary viscosity at 50° C. were determined to 101, 164, 9.2%, 37° C. and 65 mPas, respectively.

The Theological properties were compared with lanolin as described above. At the shear rate 36 1/s, the viscosity of the product of this example was 3.2 Pas, which was lower than lanolin (12.1 Pas) but higher than lanolin oil (1.1 Pas).

The hygroscopic properties of the product of this example were compared with lanolin, as described above. The product increased 3% (+1%) in weight during these two weeks. This was slightly higher than the value for lanolin (1%) but similar to lanolin oil (2% ±1%)

### Example 4

### Lipid Composition Obtained by Glycerolys of Shea Butter, Glycerol and Unsaponifiable Fraction from Shea

63 parts of refined shea butter, 10 parts of glycerol and 27 parts of the unsaponifiable fraction from shea butter were mixed at 100° C. The glycerolysis was carried out as described in Example 2. After the reaction, the resulting product was bleached and deodorised as described in Example 2, yielding a product with good adhesion and improved emulsifying properties.

The hydroxyl value, saponification value, unsaponifiable matter, slip melting point and capillary viscosity at 50° C. were determined to 99, 158, 11.5%, 41° C. and 117 mpas, respectively.

The Theological properties were compared with lanolin as described above. At the shear rate 36 1/s, the viscosity of the product of this example was 10.9 Pas, which was similar to lanolin (12.1 Pas) and higher than lanolin oil (1.1 Pas).

The hygroscopic properties of the product from this example were compared with lanolin, as described above. The product increased 5% (±1%) in weight during these two weeks. This was slightly higher than both lanolin (1%) and lanolin oil (2% ±1%).

### Example 5

### Lipid Composition Obtained be Glycerolysis of Palm Oil and Castor Oil and Subsequent Mixing with Unsaponifiable Fraction from Shea

58 parts of palm oil (Karlshamns AB, Karlshamn, Sweden), 16 parts of hydrogenated rapeseed oil with a melting point of 34° C. (Karlshamns AB, Karlshamn, Sweden), 20 parts castor oil (Björn Fredlund Ricinolja First Special Grade, Nidera, Netherlands) and 6 parts of glycerol were mixed at 100° C. Glycerolysis and bleaching/deodorisation was carried out as described in Example 2. To the resulting oily product, 5% of the unsaponifiable fraction of Example 1 was added at 90° C. before cooling the product to room temperature yielding a semisolid fat with good adhesion and emulsifying properties.

The hydroxyl value, saponification value, unsaponifiable matter, slip melting point, and capillary viscosity at 50° C. were determined to 96, 180, 1.5%, 32° C. and 56 mPas, respectively.

### Example 6

### Lipid Composition Obtained by Esterification of Shea Butter with Polyethylene Glycol and Subsequent Mixing with Unsaponifiable Fraction from Shea

In a three-necked flask 350 g of polyethylene glycol 1500 (Polyethylene glycol E1500, MBSveda, Clariant, Germany) and 650 g of shea butter were mixed. The reactants were dried for 30 minutes at 100° C. under partial vacuum (5 mbar) and gentle agitation. 3 g of NaOH were added as a catalyst. The temperature was raised to 190° C. The reaction was continued for 120 minutes with agitation and slow nitrogen. After cooling to 60° C. 500 g of the product described in Example 1(a) was added.

The hydroxyl value, saponification value, unsaponifiable matter, slip melting point and capillary viscosity at 50° C. were determined to 20, 138, 7.4%, 53° C. and 62 mPas.

### Example 7

### Lipid Composition Obtained by Esterification of Corn Oil with Glycerol and Subsequent Mixing with Unsaponifiable Fraction from Shea.

To a 1 litre four-necked flask 425 g of refined corn oil (Karlshamns AB, Karlshamn, Sweden) characterised by an iodine value of 123, and 75 g anhydrous glycerol (Tefac AB, Karlshamn, Sweden) were added. The reactants were dried at 100° C. With gentle agitation and a vacuum of 5 mbar for 30 minutes to remove residual water 1 g of sodium hydroxide (Kebo, Stockholm, Sweden) was added. The temperature was set at 160° C. for three hours to complete the reaction. The temperature was lowered to 90° C. and the reaction was quenched by addition of 10 ppm of citric acid in ethanol (20% solution). The colour was decreased with a bleaching step where 1% bleaching earth (Tonsil Optimum 215, Südchemie, Germany) was added to the flask. The bleaching procedure lasted for 30 minutes at 90° C. with agitation and a 5 mbar vacuum. After the completion of the bleaching step, 10 parts of the product of Example 1(a) was mixed with 90 parts of the glycerolised mixture. The hydroxyl value, saponification value, unsaponifiable matter, slip melting point and capillary viscosity at 50° C. were determined to 155, 150, 2.5%, 28° C., and 60 mpas, respectively.

### Example 8

### Lipid Composition from Soya Bean Oil and Shea

(a) Glycerolysis of a liquid soya bean oil

The liquid soya bean oil, characterised by an iodine value of 85 and an Unsaponifiable Content 1%, was glycerolysed with the procedure of example 1. This time the ratio oil/glycerol was 90:10 (w/w).

The hydroxyl value, saponification value, unsaponifiable matter, slip melting point and capillary viscosity at 65 C were determined to 138, 173, 0.9, 37 C and 24 cSt, respectively.

(b) Mixing of glycerolated soy bean oil and unsaponifiable from shea.

The product obtained above was mixed with the product of Example 1b (70:30) in order to obtain emulsifying properties in combination with high adhesion. The new product was bleached and deodorised by adding 1% bleaching earth (Tonsil Optimum 215 FF, Sudchemie, Germany) mixture, followed by heating to 90° C. with agitation and a 10 mbar vacuum for 30 minutes. After the completion of the bleaching step, the bleaching earth was filtered away. The product was then heated to 140° C. (3 mbar, 2 hours) and steam was flushed through the product at a rate of 36 ml/hour/kg. After cooling to 90° C., 200 ppm ascorbyl palmitate (Grindox Ascorbyl Palmitate Fine, Danisco, Brabrand, Denmark) was added to the product as an anti-oxidant. The hydroxyl value, saponification value, unsaponifiable matter, slip melting point and capillary viscosity at 50° C. were determined to 101, 164, 9.2%, 37° C. and 69 cSt, respectively.

Preparation of W/O-emulsions

Emulsions were prepared by adding a blend of demineralised water and glycerol (95:5) slowly to the oily phase at 60° C. while homogenising the emulsion using an Intermed Disp 25 (13500 rpm). The water/glycerol blend had a temperature of 50° C.

Six emulsions were prepared, three with the sample produced in this example, and three with a commercial Lanolin, as the oily phase.

Three different ratios oily phase/aqueous phase were tested (40:60, 30:70, 20:80). The results after 1 h are shown in Table 2. The emulsions were studied in a microscope to identify the phase behaviour.

TABLE 2
Stability of w/o emulsions

| | Polar | | | |
|---|---|---|---|---|
| | phase | Lanolin | Example 8 | |
| 1 | 6 g | 4 g | — | Separated in a water phase and a |
| | | | | sticky w/o-emulsion |
| 2 | 7 g | 3 g | — | Separated in a water phase and a |
| | | | | sticky w/o-emulsion |
| 3 | 8 g | 2 g | — | Separated in a water phase and a |
| | | | | sticky w/o-emulsion |
| 4 | 6 g | — | 4 g | Homogenous, w/o-emulsion |
| 5 | 7 g | — | 3 g | Homogenous, very thick w/o- |
| | | | | emulsion |
| 6 | 8 g | — | 2 g | Some water separated |

### Example 9

(a) Glycerolysis of a lipid soya bean oil and diglycerol.

A liquid soya bean oil, characterised by an iodine value of 85 and an unsaponifiable content of 1%, was glycerolysed by the procedure of Example 2. The ratio oil/diglycerol was 94:6 (w/w).

The hydroxyl value, saponification value, unsaponifiable matter, slip melting point and capillary viscosity at 65° C. were determined to 110, 173, 1.0, 33° C. and 27 cSt, respectively.

(b) Mixing of glycerolated soy bean oil and unsaponifiable from shea.

The product obtained above was mixed with product of Example 1b (80:20) in order to obtain emulsifying properties in combination with high adhesion. The new product was bleached and deodorised by adding 1% bleaching earth (Tonsil Optimum 215 FF, Südchemie, Germany) mixture, followed by heating to 90° C. with agitation and a 10 mbar vacuum for 30 minutes. After the completion of the bleaching step, the bleaching earth was filtered away. The hydroxyl value, saponification value, unsaponifiable matter, slip melting point and capillary viscosity at 50° C. were determined to 101, 164, 5.7%, 32° C. and 78 cSt, respectively.

### USE EXAMPLES

### Example 10

### Lipsticks

Red pigment (Synthetic Iron Oxide Red, Dun Chemical, Belgium) was dispersed in castor oil at 85° C. All other ingredients for each formulation were mixed at 80° C. The pigment dispersion was added at 80° C. and then the mixture was stirred until homogenous and cooled to 60° C. Finally, the lipsticks were moulded in 5 g lipstick moulds, cooled to room temperature, followed by stabilisation at 8° C. for 24 hours. The following lipstick formulations were produced and evaluated for performance, see Table 3. The figures refer to the weight of the constituent in g.

TABLE 3
Lipstick formulations

| Lipstick | | | | | | | | |
|---|---|---|---|---|---|---|---|---|
| Formulation | A | B | C | D | E | F | G | H |
| Lanolin oil* | 7.5 | — | — | — | — | — | — | — |
| Lanolin wax* | — | 7.5 | — | — | — | — | — | — |
| Example 2 | — | — | 7.5 | — | — | — | — | — |
| Example 3a | — | — | — | 7.5 | — | — | — | — |
| Example 1a | — | — | — | — | 7.5 | — | — | — |
| Example 3b | — | — | — | — | — | 7.5 | — | — |
| Example 4 | — | — | — | — | — | — | 7.5 | — |
| Example 5 | — | — | — | — | — | — | — | 7.5 |
| Beeswax | 1.8 | 1.8 | 1.8 | 1.8 | 1.8 | 1.8 | 1.8 | 1.8 |
| Microcrys- | 1.8 | 1.8 | 1.8 | 1.8 | 1.8 | 1.8 | 1.8 | 1.8 |
| talline wax | | | | | | | | |
| Carnauba wax | 1.8 | 1.8 | 1.8 | 1.8 | 1.8 | 1.8 | 1.8 | 1.8 |
| Isopropyliso | 0.9 | 0.9 | 0.9 | 0.9 | 0.9 | 0.9 | 0.9 | 0.9 |
| Stearate | | | | | | | | |
| Octyldodecan | 4.8 | 4.8 | 4.8 | 4.8 | 4.8 | 4.8 | 4.8 | 4.8 |
| ol | | | | | | | | |
| MCT oil | 2.1 | 2.1 | 2.1 | 2.1 | 2.1 | 2.1 | 2.1 | 2.1 |
| Red pigment | 2.1 | 2.1 | 2.1 | 2.1 | 2.1 | 2.1 | 2.1 | 2.1 |
| Castor oil | 6.9 | 6.9 | 6.9 | 6.9 | 6.9 | 6.9 | 6.9 | 6.9 |

*comparative examples

The following constituents were used in the lipstick formulations:
Lanolin wax from Fancor Lanolin, Fanning Corporation, USA;
Lanolin oil from Vigilan, Fanning Corporation, USA;
Beeswax from Boman Laboratories, France;
Microcrystalline wax from Deawax MW 152 DEA Mineraloel AG, Germany;
Carnauba wax from Boman Laboratories, France;
Isopropyl isostearate from Unichema, Germany;
Octyldodecanol from Eutanol G, Henkel, Germany;
MCT oil - AKOMED R, from Karlshamns AB, Sweden.

The lipstick formulations were evaluated with respect to hardness. The hardness was tested in a texture analyser (QTS25, Stevens Farnell & Co Ltd, UK) in triplicates. The results are given in Table 4 below.

TABLE 4
Evaluation of lipstick formulations

| Formulation | Hardness, kg | Standard deviation |
|---|---|---|
| A | 90 | 22 |
| B | 187 | 1 |
| C | 120 | 2 |
| D | 146 | 1 |
| E | 159 | 7 |
| F | 163 | 4 |
| G | 137 | 27 |
| H | 159 | 4 |

The lipsticks were evaluated by a test panel comprising 5 trained sensory analysts by applying the stick to the inner side of the forearm. Two sensory properties, the “touch on application” and the “film forming ability” were scored on a scale 1-5. Two commercial products Lancome 230 and Lancome 272 (Lancome, Paris, France) were used as references. Lancome 272 was perceived as a high quality lipstick and assigned a value of 5.0 on both properties while Lancome 230 was perceived as a more average type of product and assigned a value of 3.0 on both properties. The values obtained for Formulations A-H and the two reference products are given in Table 5.

TABLE 5
Sensory properties of lipstick formulations

| | | Film forming |
|---|---|---|
| Formulation | Touch | properties |
| A | 4.0 | 3.5 |
| B | 3.5 | 2.0 |
| C | 3.0 | 4.5 |
| D | 4.0 | 4.5 |
| E | 3.0 | 3.0 |
| F | 4.0 | 4.5 |
| G | 3.5 | 3.5 |
| H | 3.5 | 4.0 |
| Lancome 272* | 5.0 | 5.0 |
| Lancome 230* | 3.0 | 3.0 |

Formulations A, D and G were further evaluated on the lips. In a blind test six females applied the lipsticks during normal conditions. Certain tests were added, such as test of the products when drinking coffee in white cups. Each lipstick was scored (1-5). The touch when applying lipstick G on the lips were similar to the lanolin oil based lipstick. However, lipstick G felt less smeary and kept the shape much better than the other two formulations. Also the spreading on the lips appeared to be more uniform. The results are given in the following Table 6.

TABLE 6
Evaluation of lipstick formulations in vivo

| Formulation | A | D | G |
|---|---|---|---|
| Touch when applied | 3.7 | 4.2 | 3.8 |
| Smeariness when applied | 3.2 | 4.0 | 2.7 |
| Ability to stay in place | 3.5 | 3.2 | 4.0 |
| Even spreading of colour | 3.8 | 3.7 | 4.3 |

### Example 13

### Creams

The performance of the compositions obtained in Example 3(a) and 3(b) in cosmetic emulsions were evaluated by producing four creams according to the recipes in Table 6 below. Emulsions were prepared by mixing the oily phase ingredients at 70° C., heating the water (with or without the Carbopol) to 70° C. and slowly adding the oil phase to the water while stirring. After pH adjustment, the emulsions were homogenised for 1 minute using an Ultra-Turrax homogeniser, cooled to about 40° C. and bottled. The formulations were evaluated for stability at 40° C. and were found to be stable for more than 1 month without signs of separation.

TABLE 7

| | Formu- | Formu- | Formu- | Formu- |
|---|---|---|---|---|
| | lation 1 | lation 2 | lation 3 | lation 4 |
| Oil phase | | | | |
| Example 3a | 5.0% | — | — | — |
| Example 3b | — | 5.0% | — | 50% |
| Lanolin | | | 5.0% | |
| Rylo CI25 | 4.0% | 4.0% | 4.0% | — |
| AKOMED R | 10.0% | 10.0% | 10.0% | — |
| Aqueous phase | | | | |
| Distilled | 81.0% | 80.7% | 80.7% | 50 |
| water | | | | |
| Carbopol | — | 0.3% | 0.3% | — |
| pH (adjusted | 6.9 | 6.2 | 6.4 | 7.0 |
| with NaOH_{aq}) | (No | | | (No |
| | adjustment | | | adjustment |
| | needed) | | | needed) |

The formulations were evaluated by a group of five persons (three females and two males). Formulation 1 resulted in a thin lotion, and Formulation 2 in a creamy o/w-emulsion with a hint of stickiness. Formulation 3 was very similar to Formulation 2. However, the smell of lanolin could be recognised; the other formulations were odourless. Formulation 4 (w/o-emulsion) was very fatty and tacky. All formulations were completely homogenous and white.

### Example 12

### Shampoo

To evaluate if the compositions of the invention are suitable for hair-care applications, shampoo formulations were produced (100 g each), Table 8. The ingredients B-G were mixed and in turn added to the distilled water. The sodium chloride was then mixed in and the pH was adjusted. While the reference, Formulation 2, was clear, the other four shampoos had a creamy appearance.

TABLE 8
Shampoo formulations

| Ingredient | | 1 | | 2 | | 3 | | 4 | | 5 | |
|---|---|---|---|---|---|---|---|---|---|---|---|
| A | Distilled water | 54 | g | 54 | g | 55 | g | 54 | g | 54 | g |
| B | Example 3b | 2 | g | — | | — | | — | | — | |
| C | Example 5 | — | | — | | 2 | g | — | | — | |
| D | Texapon NSO | 27 | g | 27 | g | 27 | g | 27 | g | 27 | g |
| E | Plantcare 818 UP | 9 | g | 9 | g | 9 | g | 9 | g | 9 | g |
| F | Mackam CB-35 | 6 | g | 6 | g | 6 | g | 6 | g | 6 | g |
| G | AKOMED R* | — | | — | | — | | 2 | g | — | |
| H | Lanolin* | — | | — | | — | | — | | 2 | g |
| I | Sodium chloride | 1 | g | 1 | g | 1 | g | 1 | g | 1 | |
| | pH adjusted to | 5.3 | | 5.4 | | 5.4 | | 5.4 | | 5.5 | |

*Comparative example
Texapon NSO from Cognis, Germany
Plantcare 818 UP from Henkel, Germany
Mackam CB-35 from McIntyre Group Ltd, USA
AKOMED R, a MCT-oil from Karlshamns AB, Sweden

A study was performed to evaluate the foaming properties of the shampoo formulations in a 100 ml scale. 10 g of each shampoo was weighed in a 300 ml beaker. Water (40° C.) was added gently. An electric hand-mixer was used to create the foam. The maximum speed was used for 60 seconds to create foam. The height of the foam was measured directly after the agitation, and after two hours, see Table 9. Formulation 2 was used as reference and the other formulations are indexed based on said reference. The values refer to the height of the foam in mm. Each shampoo was tested twice.

TABLE 9
Foam height in % of Formulation 2

| | | Shampoo | | | | | | | | | |
|---|---|---|---|---|---|---|---|---|---|---|---|
| | | 1 | | 2 | | 3 | | 4 | | 5 | |
| | | Mean | | Mean | | Mean | | Mean | | Mean | |
| | | value | ± | value | ± | value | ± | value | ± | value | ± |
| 1 | min | 110 | 4 | 100 | 12 | 118 | 3 | 89 | 4 | 116 | 2 |
| 120 | min | 108 | 4 | 100 | 20 | 108 | 4 | 88 | 4 | 108 | 4 |

The results clearly show that the Formulations 1 and 3 according to the invention, as well as Formulation 5 with lanolin, do not inhibit foaming, and therefore are useful in hair-care formulations with re-fattening properties. The addition of AKOMED R, a medium chain glyceride, seems to have an inhibiting effect on the foaming.

### PHYSICAL EVALUATION OF THE LIPID COMPOSITIONS OF THE EXAMPLES AND REFERENCE MATERIALS

Water absorption properties

The emulsifying properties were studied by adding 1 g of each substance to 5-ml test tubes. The samples were heated to 70° C. for 30 minutes and water (40° C.) was added in 0.5, 0.75, 1, 1.25 or 1.5 g additions. The mixtures were then rapidly shaken in a shaker (vibromix, Whirli Vib 1) at 2000 rpm for 10 seconds. The maximum amount of water, which resulted in a homogenous emulsion after 72 hours of storage at 30° C. was taken as a measure of the emulsifying capacity. The results are given in Table 10.

TABLE 10
Emulsified amount of water in % by weight

| | Maximum emulsified |
|---|---|
| Composition | amount of water |
| Lanolin* | 100% |
| Lanolin** | 75% |
| Example 2 | 100% |
| Example 1 | 0% |
| Example 3 (a) | 100% |
| Example 3 (b) | 100% |
| Example 4 | 150% |
| Example 5 | 125% |
| Kahls Lanolin substitute | <50%, |
| | some emulsifying effect |
| Example 6 | 150% |
| Example 7 | 125% |
| Example 8b | 125% |
| Example 9b | 75% |
| Shea butter | 0% |
| Soy bean oil | 0% |

*Fancor Lanolin from Fanning Corporation, USA
**Lanolin Anhydrous, BDH Laboratory suppliers, UK
Shea butter from Karlshamns AB, Karlshamn, Sweden
Soya bean oil from Karlshamns AB, Karlshamn, Sweden

These results show that neither the non-polar starting material described in Example 1 nor the triglyceride oils have the capacity to emulsify water.

Neither shea butter nor soya bean oil have any similarities to lanolin with respect to emulsifying properties.

Adhesiveness

The adhesiveness of the compositions of the Examples and some reference products was analysed in a texture analyser (QTS25, Stevens Farnell & Co Ltd, UK) in triplicates. Samples (room temperature) of the different compositions were spread on a plastic plate at a temperature of 35° C. with a 1-mm dent (diameter 20 mm). A probe (diameter 8 mm) of stainless steel entered the samples from above with a speed of 30 mm/min and stopped when a load of 300 g was reached. Then the probe returned to its initial position. The program was set to repeat the procedure. The adhesiveness is correlated to the force used for the probe to return to its initial position, i.e. area under curve (working force in g.s). Each sample was spread on three different plates and analysed. The results are given in Table 11. The results show that commercial mono- and diglycerides lack the adhesiveness associated with lanoline.

TABLE 11

| | Adhesiveness, | |
|---|---|---|
| Product | gram · second | Standard deviation |
| Lanolin* | 39.2 | 5.0 |
| Lanolin** | 24.2 | 2.9 |
| Maisine | 3.3 | 1.4 |
| Example 2 | 101.0 | 12.9 |
| Example 3a | 14.9 | 1.3 |
| Example 1a | 17.3 | 1.1 |
| Example 3b | 31.1 | 2.1 |
| Example 4 | 40.2 | 6.2 |
| Example 5 | 12.2 | 5.3 |
| Example 6 | 62.8 | 1.6 |
| Example 7 | 6.4 | 2.1 |

*Fancor Lanolin from Fanning Corporation, USA
**Lanolin Anhydrous, BDH Laboratory suppliers, UK
Maisine 35, mono- and diglyceride from Gattefossé,
France all used for comparative purposes

The same procedure was repeated at a temperature of 20° C. with a probe having a diameter of 10 mm, which entered the samples from above with a speed of 10 mm/min and stopped when a load of 300 g was reached. The results of this second test are given in Table 12.

TABLE 12

| | Adhesiveness, |
|---|---|
| Product | gram · second |
| Lanolin* | 297 |
| Lanolin** | 160 |
| Acetylated lanolin*** | 101 |
| Maisine | 32 |
| Shea butter | 43 |
| Example 1a | 66 |
| Example 2 | 43 |
| Example 3a | 322 |
| Example 3b | 436 |
| Example 4 | 535 |
| Example 5 | 492 |
| Example 6 | 629 |
| Example 7 | 58 |
| Example 8b | 165 |

*Fancor Lanolin from Fanning Corporation, USA
**Lanolin Anhydrous, BDH Laboratory suppliers, UK
***Fancor Acetylated Lanolin from Fanning Corporation, USA
Maisine 35, mono- and diglyceride from Gattefossé, France
Shea butter, Karlshamns AB. Sweden
all used for comparative purposes

The results show that neither commercial mono- and diglycerides, nor triglycerides such as shea butter have an adhesiveness comparable with lanoline.

The above tests also show that twhe temperature has a large impact on the adhesiveness of the lipid compositions, and explains the differences of the individual samples at 20° C. and 35° C., respectively. The products of Examples 2-6, and 8b all have melting points within the range of 30-55° C.

In order to understand the effect when mixing the polar componenent with non-polar unsaponifiable matter, the adhesiveness of different blends of the product of Example 6 and the product of Example la-were also investigated at 20° C. The results are given in Table 13 below.

TABLE 13

| | Adhesiveness | |
|---|---|---|
| | (gram · seconds) | Unsaponifiables |
| Example 7 without | 19 | <1% |
| addition of sample | | |
| from Example 1a | | |
| Example 7 | 58 | 2.5% |
| Example 7 + Example | 86 | 5% |
| 1a (90:10) | | |
| Example 7 + Example | 137 | 9% |
| 1a (70:30) | | |

This shows that the adhesiveness obtained exceeds the values of the individual adhesiveness.
Sensory properties

The smell and taste of lanolin and the compositions of Examples 3(a), 3(b), 4 and 5 were tested by a sensory panel, with long experience of testing vegetable oils and fats for edible uses. Samples of the compositions were heated to 40° C. and served in random order to a panel consisting of 5 experienced sensory analysts. 6 different characteristics were scored on a non-marked linear scale from 0 to 10 and the average scores were calculated after removal of obvious outliers. All the samples were stored for two months after production at 8° C. in glass bottles before evaluation. Lanolin samples were evaluated as received. The results are presented in Table 14.

TABLE 14

| | | Smell | | Hydro- | | | |
|---|---|---|---|---|---|---|---|
| | Strong | of | Neutral | gena- | | | |
| Product | Smell | wool | taste | tion | Sweet | Bitter | Pungent |
| Lanolin* | 9.0 | 7.7 | 1.0 | 1.0 | 1.0 | 9.0 | 8.3 |
| Example | 2.0 | 1.2 | 6.3 | 2.0 | 4.1 | 1.7 | 1.6 |
| 3 (a) | | | | | | | |
| Example | 2.7 | 1.5 | 6.0 | 3.3 | 4.1 | 1.7 | 2.0 |
| 3 (b) | | | | | | | |
| Example 4 | 2.0 | 1.2 | 6.0 | 4.0 | 4.0 | 1.0 | 2.0 |
| Example 5 | 1.6 | 1.0 | 8.1 | 2.3 | 2.7 | 1.6 | 1.5 |

*Fancor lanolin from Fanning Corporation, USA

In order to compare the touch, adhesion and ability to form a film on the skin, the test panel (six females) evaluated samples from Examples 2, 3(a), 3(b) and 4. The products were scored from 0 to 5. A lanolin creme containing solely lanolin and purified water (75:25) and a mono-diglyceride were used as references.

The samples as well as the mono-diglyceride reference were prepared to imitate the lanolin crème. 30 g of each sample were heated to 70° C. and then 10 g of distilled water (40° C.) were added. In turn each sample was then homogenised at 20500 rpm (Heidolf Diax 600, Labasco, Sweden). The samples were stored at room temperature for one week before being evaluated on the skin. The results are given in Table 15 below.

TABLE 15

| | | | Film forming | Oily | Gloss on |
|---|---|---|---|---|---|
| Product | Touch | Adhesion | properties | afterfeel | the skin |
| Lanolin* | 5.0 | 5 | 5.0 | 0.0 | 5.0 |
| Mono-di- | 0.0 | 0.0 | 1.0 | 4.0 | 1.0 |
| Glyceride** | | | | | |
| Example 2 | 4.0 | 4.0 | 3.5 | 1.0 | 2.5 |
| Example | 1.0 | 1.0 | 1.8 | 4.8 | 4.5 |
| 3 (a) | | | | | |
| Example | 2.0 | 2.0 | 2.6 | 3.5 | 2.0 |
| 3 (b) | | | | | |
| Example 4 | 3.3 | 3.3 | 3.5 | 2.0 | 4.0 |

*Lanolin ATL-K, Apoteksbolaget, Sweden
**Maisine 35, mono- and diglyceride from Gattefossé, France used for comparison

### CONCLUSIONS

Lanolin-like compositions based on a combination of unsaponifiable matter with a polar mixture of fatty esters are described in Examples 1-9 and their uses in various applications in Examples 10-12. The unsaponifiable matter comprises a mixture of long-chain hydrocarbons and sterol or triterpene alcohols and their esters. The polar fatty esters are primarily glycerides or polyethylene glycol esters. The properties of the compositions may be varied in a wide range depending on the intended application. The adhesiveness or tackiness of the composition is controlled by the amount of the unsaponifiable fraction added and by the degree of saturation and esterification of the fatty acid esters.

Polarity, expressed in water absorption and emulsifying capacity, is primarily controlled by the degree of esterification, conveniently expressed as the hydroxyl value.

1. A lipid composition, in comprising
(i) a non-polar unsaponifiable matter precipitated from one or more vegetable oils and fats selected from the group consisting of shea butter, olive oil, avocado oil and mixtures thereof, in an amount not less than 2% by weight of said composition, and
(ii) a polar mixture of fatty acid esters derived from alcoholysis of triglycerides with glycerol, polyglycerols or polyethylene glycols,
wherein said composition consists of 5-33% by weight of a mixture of said unsaponifiable matter (i) and a vegetable oil, and 67-95% by weight of said polar mixture (ii), wherein said composition has a lanolin-like property an adhesiveness, and a water absorption of at least 50% by weight.

2. The lipid composition according to claim 1, wherein said unsaponifiable matter is derived from crude or refined shea butter.

3. The lipid composition according to claim 1, wherein said unsaponifiable matter is precipitated from said vegetable oil by addition of acetone.

4. The lipid composition according to claim 1, wherein said unsaponifiable matter comprises long-chain hydrocarbons, sterols, triterpene alcohols, and esters of sterols and triterpene alcohols.

5. The lipid composition according to claim 1, wherein said fatty acid esters are derived from straight or branched, saturated or unsaturated C8 to C24 fatty acids or hydroxy fatty acids.

6. The lipid composition according to claim 1 wherein said polar mixture of fatty acid esters is derived from a vegetable oil.

7. The lipid composition according to claim 6, wherein said vegetable oil is selected from the group consisting of soybean oil, corn oil, and mixtures thereof.

8. The lipid composition according to claim 1, having a hydroxyl value of 20-180, a slip melting point of 30-55° C., and a water absorption of 75-200% by weight.

9. The lipid composition according to claim 1, wherein the content of said unsaponifiable matter is 2-25% by weight of said composition.

10-13. (canceled)

14. A method of preparing a formulation, said method comprising substituting a lanolin comprised in said formulation with said lipid composition as claimed in claim 1.

15. The method as claimed in claim 14, wherein said formulation is a pharmaceutical formulation further comprising one or more conventional additives and one or more active substances.

16. The method as claimed in claim 14, wherein said formulation is a cosmetic formulation further comprising one or more conventional additives and one or more active substances.

17. The method as claimed in claim 16, wherein said cosmetic formulation is capable of being utilized as one or more colour cosmetics, toiletries, skin care, hair care, and bath and shower products.
